(19) United States
(12) Patent Application Publication
(10) Pub. No.: US 2011/0172672 A1
(43) Pub. Date: Jul. 14, 2011

(54) INSTRUMENT WITH TRANSPARENT PORTION FOR USE WITH PATIENT-SPECIFIC ALIGNMENT GUIDE

(75) Inventors: Serge Dubeau, Plymouth, MN (US); Robert Metzger, Wakarusa, IN (US)

(73) Assignee: BIOMET MANUFACTURING CORP., Warsaw, IN (US)

(21) Appl. No.: 13/045,169

(22) Filed: Mar. 10, 2011

Related U.S. Application Data
(63) Continuation-in-part of application No. 12/973,214, filed on Dec. 20, 2010.
Application No. 12/973,214 is a continuation-in-part of application No. 12/955,361, filed on Nov. 29, 2010.
Application No. 12/955,361 is a continuation-in-part of application No. 12/938,905, filed on Nov. 3, 2010.
Application No. 12/938,905 is a continuation-in-part of application No. 12/938,913, filed on Nov. 3, 2010.
Application No. 12/938,905 is a continuation-in-part of application No. 12/893,306, filed on Sep. 29, 2010.
Application No. 12/938,913 is a continuation-in-part of application No. 12/893,306, filed on Sep. 29, 2010.
Application No. 12/893,306 is a continuation-in-part of application No. 12/888,005, filed on Sep. 22, 2010.
Application No. 12/888,005 is a continuation-in-part of application No. 12/714,023, filed on Feb. 26, 2010.
Application No. 12/714,023 is a continuation-in-part of application No. 12/571,969, filed on Oct. 1, 2009.
Application No. 12/571,969 is a continuation-in-part of application No. 12/486,992, filed on Jun. 18, 2009.
Application No. 12/486,992 is a continuation-in-part of application No. 12/389,901, filed on Feb. 20, 2009.
Application No. 12/389,901 is a continuation-in-part of application No. 12/211,407, filed on Sep. 16, 2008.
Application No. 12/211,407 is a continuation-in-part of application No. 12/039,849, filed on Feb. 29, 2008.
Application No. 12/039,849 is a continuation-in-part of application No. 11/756,057, filed on May 31, 2007.
Application No. 12/039,849 is a continuation-in-part of application No. 11/971,390, filed on Jan. 9, 2008.
Application No. 11/971,390 is a continuation-in-part of application No. 11/363,548, filed on Feb. 27, 2006, now Pat. No. 7,780,672.
Application No. 12/039,849 is a continuation-in-part of application No. 12/025,414, filed on Feb. 4, 2008.
Application No. 12/025,414 is a continuation-in-part of application No. 12/872,663, filed on Aug. 31, 2010.
Application No. 12/872,663 is a continuation-in-part of application No. 12/483,807, filed on Jun. 12, 2009.
Application No. 12/483,807 is a continuation-in-part of application No. 12/371,096, filed on Feb. 13, 2009.
Application No. 12/371,096 is a continuation-in-part of application No. 12/103,824, filed on Apr. 16, 2008.
Application No. 12/103,824 is a continuation-in-part of application No. 12/103,834, filed on Apr. 16, 2008.
Application No. 12/103,834 is a continuation-in-part of application No. 12/978,069, filed on Dec. 23, 2010.
Application No. 12/978,069 is a continuation-in-part of application No. 12/973,214, filed on Dec. 20, 2010.
Application No. 12/973,214 is a continuation-in-part of application No. 13/041,469, filed on Mar. 7, 2011.
Application No. 13/041,469 is a continuation-in-part of application No. 13/041,495, filed on Mar. 7, 2011.
Application No. 13/041,495 is a continuation-in-part of application No. 13/041,665, filed on Mar. 7, 2011.
Application No. 13/041,665 is a continuation-in-part of application No. 13/041,883, filed on Mar. 7, 2011.
(60) Provisional application No. 61/446,660, filed on Feb. 25, 2011.
(60) Provisional application No. 60/953,620, filed on Aug. 2, 2007.
(60) Provisional application No. 60/947,813, filed on Jul. 3, 2007.
(60) Provisional application No. 60/911,297, filed on Apr. 12, 2007.
(60) Provisional application No. 60/892,349, filed on Mar. 1, 2007.
(60) Provisional application No. 60/812,694, filed on Jun. 9, 2006.
(60) Provisional application No. 60/953,637, filed on Aug. 2, 2007.
(60) Provisional application No. 61/310,752, filed on Mar. 5, 2010.
(60) Provisional application No. 60/912,178, filed on Apr. 17, 2007.
(60) Provisional application No. 60/912,178, filed on Apr. 17, 2007.

(51) Int. Cl. A61F 5/00

(52) U.S. Cl. 606/87

(57) ABSTRACT

A system for orienting objects relative to an anatomical feature of a patient includes a guide with a reference guide surface. The system also includes a referencing object that is guided by the reference guide surface toward the anatomical feature to be fixed to the anatomical feature in a reference orientation relative to the anatomical feature. Moreover, the system includes an instrument with at least a portion that is transparent. The referencing object is viewable through the transparent portion of the instrument to align the referencing object with the instrument and to orient the instrument in the reference orientation relative to the anatomical feature.

### CROSS-REFERENCE TO RELATED APPLICATIONS

This application claims the benefit of U.S. Provisional Application No. 61/446,660, filed on Feb. 25, 2011.

This application is a continuation-in-part of U.S. application Ser. Nos. 13,041,469, 13/041,495, 13/041,665 and 13/041,883, each filed on Mar. 7, 2011, each of which is a continuation-in-part of U.S. application Ser. No. 12/978,069 filed Dec. 23, 2010, which is a continuation-in-part of U.S. application Ser. No. 12/973,214, filed Dec. 20, 2010, which is a continuation-in-part of U.S. application Ser. No. 12/955,361 filed Nov. 29, 2010, which is a continuation-in-part of U.S. application Ser. Nos. 12/938,913 and 12/938,905, each filed on Nov. 3, 2010, each of which is a continuation-in-part of U.S. application Ser. No. 12/893,306, filed Sep. 29, 2010, which is a continuation-in-part of U.S. application Ser. No. 12/888,005, filed Sep. 22, 2010, which is a continuation-in-part of U.S. application Ser. No. 12/714,023, filed Feb. 26, 2010, which is a continuation-in-part of U.S. application Ser. No. 12/571,969, filed Oct. 1, 2009, which is a continuation-in-part of U.S. application Ser. No. 12/486,992, filed Jun. 18, 2009, and is a continuation-in-part of U.S. application Ser. No. 12/389,901, filed Feb. 20, 2009, which is a continuation-in-part of U.S. application Ser. No. 12/211,407, filed Sep. 16, 2008, which is a continuation-in-part of U.S. application Ser. No. 12/039,849, filed Feb. 29, 2008, which: (1) claims the benefit of U.S. Provisional Application No. 60/953,620, filed on Aug. 2, 2007, U.S. Provisional Application No. 60/947,813, filed on Jul. 3, 2007, U.S. Provisional Application No. 60/911,297, filed on Apr. 12, 2007, and U.S. Provisional Application No. 60/892,349, filed on Mar. 1, 2007; (2) is a continuation-in-part U.S. application Ser. No. 11/756,057, filed on May 31, 2007, which claims the benefit of U.S. Provisional Application No. 60/812,694, filed on Jun. 9, 2006; (3) is a continuation-in-part of U.S. application Ser. No. 11/971,390, filed on Jan. 9, 2008, which is a continuation-in-part of U.S. application Ser. No. 11/363,548, filed on Feb. 27, 2006, now U.S. Pat. No. 7,780,672, issued Aug. 24, 2010; and (4) is a continuation-in-part of U.S. application Ser. No. 12/025,414, filed on Feb. 4, 2008, which claims the benefit of U.S. Provisional Application No. 60/953,637, filed on Aug. 2, 2007.

U.S. application Ser. No. 12/938,913, filed Nov. 3, 2010 and U.S. application Ser. No. 12/938,905, filed Nov. 3, 2010, are also each a continuation-in-part of U.S. application Ser. No. 12/872,663, filed on Aug. 31, 2010, which claims the benefit of U.S. Provisional Application No. 61/310,752 filed on Mar. 5, 2010.

U.S. application Ser. No. 12/938,913, filed Nov. 3, 2010 and U.S. application Ser. No. 12/938,905, filed Nov. 3, 2010, are also each a continuation-in-part of U.S. application Ser. No. 12/483,807, filed on Jun. 12, 2009, which is a continuation-in-part of U.S. application Ser. No. 12/371,096, filed on Feb. 13, 2009, which is a continuation-in-part of U.S. application Ser. No. 12/103,824, filed on Apr. 16, 2008, which claims the benefit of U.S. Provisional Application No. 60/912,178, filed on Apr. 17, 2007.

U.S. application Ser. No. 12/938,913, filed Nov. 3, 2010 and U.S. application Ser. No. 12/938,905, filed Nov. 3, 2010, are also each a continuation-in-part of U.S. application Ser. No. 12/103,834, filed on Apr. 16, 2008, which claims the benefit of U.S. Provisional Application No. 60/912,178, filed on Apr. 17, 2007.

The disclosures of the above references are incorporated herein by reference.

### FIELD

The following relates to an instrument and, more particularly, relates to an instrument with a transparent portion for use with a patient-specific alignment guide.

### INTRODUCTION

It is known to attach various types of instruments to anatomical features using pins or other referencing objects. For instance, one or more pins can be attached to a bone at predetermined locations, and a resection guide can slide over and receive the pin(s) to attach the resection guide to the bone. Then, a cutting tool, such as a reciprocating blade, etc. can be guided by the resection guide to cut and resect the bone in preparation of implanting a prosthetic device to the bone.

In some cases, aligning the instrument to the referencing object can be difficult because the instrument can block the surgeon's view of the referencing object. Accordingly, the surgeon may need to view the pins and instrument from the side, from a viewpoint that is transverse to the axis of alignment, while attempting to align these objects. However, other objects (e.g., the patient's anatomy and/or other instruments) may further limit the surgeon's view. The surgeon can also attempt to align the objects using the sense of touch; however, this can be inaccurate. As such, aligning the instrument to the pin(s) can be cumbersome and inconvenient for the surgeon.

### SUMMARY

A cutting guide for cutting an anatomical feature is disclosed. The cutting guide includes a first portion with a cutting guide surface that guides a cutting device when cutting the anatomical feature. The first portion is opaque. The cutting guide also includes a second portion that is coupled to the first portion. The second portion is transparent for viewing the anatomical feature through the second portion and for orienting the cutting guide relative to the anatomical feature.

A system for orienting objects relative to an anatomical feature of a patient is additionally disclosed. The system includes a guide with a reference guide surface. The system also includes a referencing object that is guided by the reference guide surface toward the anatomical feature to be fixed to the anatomical feature in a reference orientation relative to the anatomical feature. Moreover, the system includes an instrument with an opening and at least a portion that is transparent adjacent the opening. The referencing object is viewable through the transparent portion of the instrument to align the referencing object with the opening. The opening receives the referencing object to orient the instrument in the reference orientation relative to the anatomical feature.

Furthermore, a method of orienting objects relative to an anatomical feature of a patient is disclosed. The method includes coupling a guide to the anatomical feature. The method also includes guiding a referencing object toward the anatomical feature using a reference guide surface of the guide to establish the referencing object in a reference orientation relative to the anatomical feature. Additionally, the method includes providing an instrument with an opening and at least a portion that is transparent adjacent the opening. Also, the method includes orienting the instrument in the reference orientation relative to the anatomical feature by viewing the referencing object through the portion of the instrument and receiving the referencing object with the opening of the instrument.

Still further, a system for orienting objects relative to an anatomical feature of a patient is disclosed. The system includes a patient-specific guide having a patient-specific surface with a three-dimensional contour that nests and closely conforms to a corresponding surface of the anatomical feature to align the patient-specific guide relative to the anatomical feature. The patient-specific guide further includes a reference guide surface. The system also includes a pin that is guided by the reference guide surface toward the anatomical feature to be fixed to the anatomical feature in a reference orientation relative to the anatomical feature. Moreover, the system includes a cutting guide with an opening. The cutting guide includes a metallic portion that defines a cutting guide surface that guides a cutting device for cutting the anatomical feature. The cutting guide also includes a transparent portion that defines the opening. The pin is viewable through the transparent portion to align the pin with the opening, and the opening receives the pin to orient the cutting guide in the reference orientation relative to the anatomical feature.

Further areas of applicability of the present teachings will become apparent from the description provided hereinafter. It should be understood that the description and specific examples are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIG. 1 is an exploded perspective view of a system for orienting objects relative to an anatomical feature according to various teachings of the present disclosure;

FIG. 2 is a perspective view of various components of the system of FIG. 1;

FIG. 3 is a perspective view of various other components of the system of FIG. 1;

FIG. 4 is a front, perspective view of a cutting guide with a transparent portion of the system of FIG. 1;

FIG. 5 is a rear, perspective view of the cutting guide of the system of FIG. 1;

FIG. 6 is perspective view of the cutting guide of FIGS. 4 and 5 being used in an alternative embodiment;

FIG. 7 is a perspective view of another embodiment of the cutting guide;

FIG. 8 is a front view of an instrument with a transparent portion of the system of FIG. 1 according to various other exemplary embodiments; and

FIG. 9 is a perspective view of an instrument with a transparent portion of the system of FIG. 1 according to various other exemplary embodiments.

### DESCRIPTION OF VARIOUS ASPECTS

The following description is merely exemplary in nature and is in no way intended to limit the present teachings, applications, or uses. For example, although the present teachings are illustrated for alignment guides, resection guides, and instruments for performing knee surgery (e.g., knee arthroplasty), the present teachings can be used for other guides, templates, jigs, drills, rasps or other instruments used in various orthopedic procedures. Moreover, although patient-specific alignment guides are illustrated for use with either standard or patient-specific transparent instruments, it will be appreciated that the transparent instruments could be used with non-patient specific guides without departing from the scope of the present disclosure.

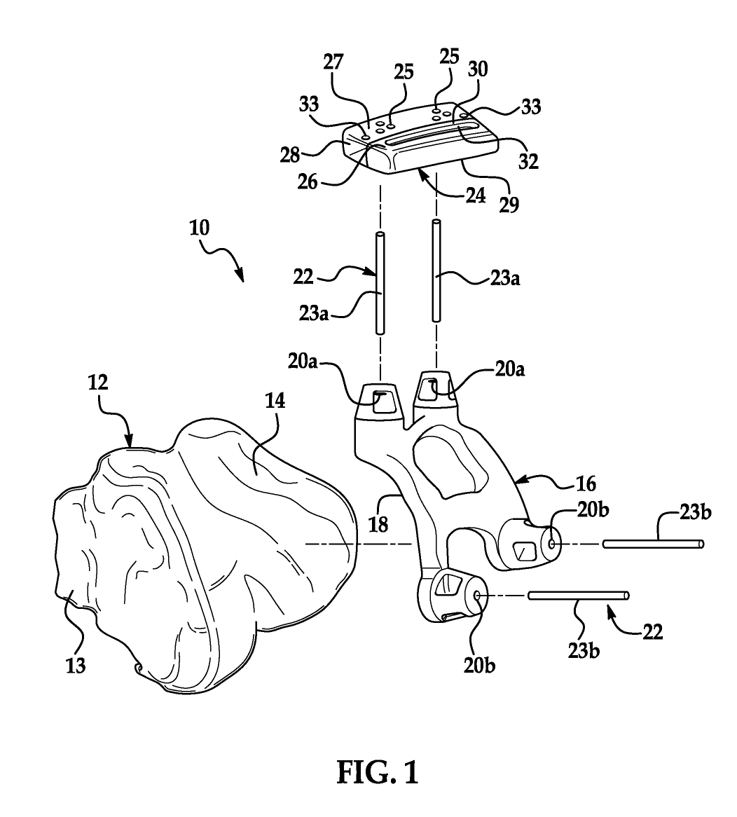

Referring initially to FIG. 1, a system 10 for orienting objects relative to an anatomical feature 12 is illustrated. In the embodiments illustrated, the system 10 is used in connection with knee surgery (e.g., resecting a femur 13 and/or a tibia 40′ (FIG. 6) for implanting a knee joint prosthetic assembly). However, it will be appreciated that the system 10 can be used for orienting objects on any suitable anatomical feature 12 without departing from the scope of the present disclosure.

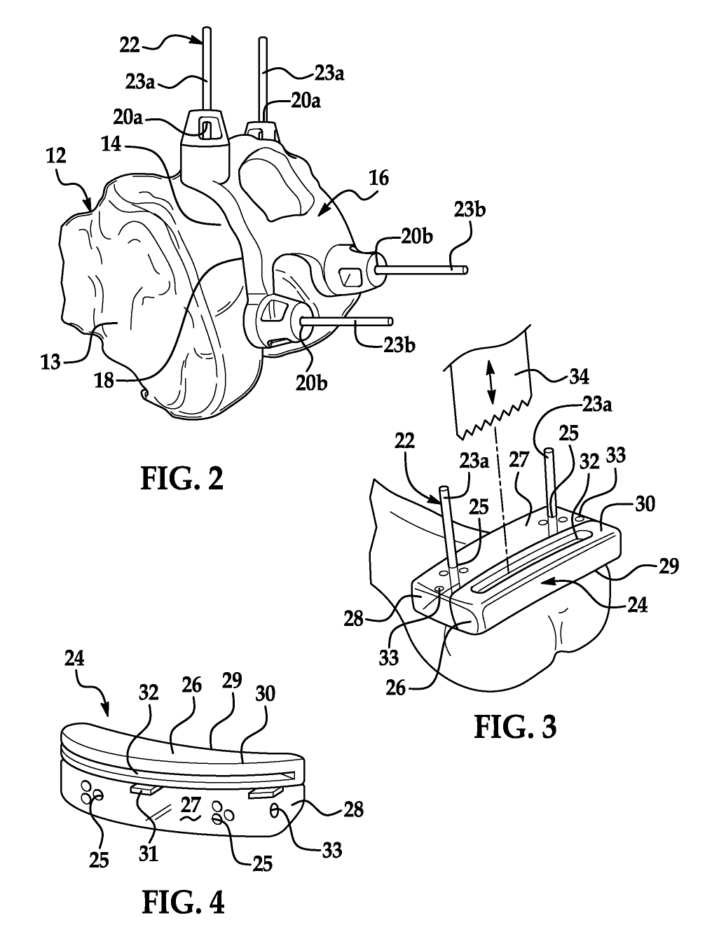

Generally, the system 10 can include a patient-specific alignment guide 16 with a three-dimensional patient-specific surface 18 that nests and closely conforms to a corresponding surface 14 of the anatomical feature 12 to align the guide 16 relative to the anatomical feature 12 in a single orientation. The alignment guide 16 can also include one or more reference guide surfaces 20a, 20b (e.g., inner surface of a through-hole). The system 10 can also include one or more referencing objects 22 (pins, nails, etc.) that are each guided by a corresponding reference guide surface 20a, 20b of the alignment guide 16 toward the anatomical feature 12 to be fixed to the anatomical feature 12 in a reference orientation relative to the anatomical feature 12 (FIG. 2). Still further, the system 10 can include an instrument 24, such as a cutting guide 26 for resecting the anatomical feature 12. The instrument 24 can include one or more reference openings 25 that receive a corresponding referencing object 22. Thus, the instrument 24 can be attached to the anatomical feature 12 in the reference orientation by moving the instrument 24 onto the referencing object(s) 22 (FIG. 3).

As will be discussed, the instrument 24 can be at least partially transparent to aid the user in positioning the instrument 24 on the referencing object(s) 22. More specifically, the instrument 24 can be transparent adjacent the reference openings 25. As such, the referencing object 22 can be viewed through the transparent portion of the instrument 24 to aid in aligning the referencing object 22 with the corresponding reference opening 25. Accordingly, the instrument 24 can be visually aligned with the referencing object(s) 22 more quickly and more conveniently instead of merely relying on the sense of touch for aligning the instrument 24 to the object(s) 22 and/or instead of viewing the instrument 24 and referencing object(s) 22 transversely from the axis of alignment. Thus, the overall medical procedure can be less time consuming and more convenient.

Referring now to FIGS. 1 and 2, the patient-specific alignment guide 16 will be discussed in greater detail. The alignment guide 16 can have any suitable shape and can be made out of any suitable material (e.g., a rigid polymeric material). The alignment guide 16 can also include any suitable guide surface 20a, 20b for guiding the referencing objects 22. For instance, the guide surface 20a, 20b can be the inner surface of a rounded through-hole as shown in FIG. 2; however, the guide surface 20a, 20b can be a groove, a notch, another peripheral surface of the alignment guide 16, etc. Also, the alignment guide 16 can include any suitable number of guide surfaces 20a, 20b. In the embodiments illustrated, the alignment guide 16 includes four guide surfaces 20a, 20b, wherein two of the guide surfaces 20a are intended to be disposed over the anterior of the anatomical feature 12 and wherein the other two guide surfaces 20b are intended to be disposed over the distal end of the anatomical feature 12. However, the alignment guide 16 can include more or less than four guide surfaces 20a, 20b, and the guide surfaces 20a, 20b can be disposed over any portion of the anatomical feature 12.

Moreover, the patient-specific surface 18 can have a three-dimensional contour that nests and closely conforms to the corresponding surface 14 of the anatomical feature 12 in a single orientation. For instance, the patient-specific surface 18 can extend over a distal and anterior surface 14 of the femur 13. As such, the alignment guide 16 can nest on the cartilage surface 14 of the femur 13 as shown in FIG. 3 such that the guide surfaces 20a, 20b are disposed in a predetermined orientation relative to the distal end of the femur 13.

The alignment guide 16 can be generated in various ways. For instance, the dimensions, geometry, and other features of the patient's anatomical femur 13 can be detected using a suitable imaging device (X-ray, CT scan, MRI, etc.) before surgery. Then, an electronic model can be generated, and the alignment-guide 16 can be fabricated according to that electronic model with the patient-specific surface 18 and guide surfaces 20a, 20b included thereon.

In some embodiments, the alignment guide 16 and its method of manufacture can incorporate various features disclosed in commonly-owned, co-pending in U.S. patent application Ser. No. 11/756,057, filed on May 31, 2007, incorporated herein by reference.

Moreover, the patient-specific alignment guide 16 can be used either with conventional or patient-specific instruments and prosthetic implant components. The patient-specific instruments and prosthetic implant components can be prepared with computer-assisted imaging methods. For example, computer modeling for obtaining three dimensional images of the patient's anatomy using MRI or CT scans of the patient's anatomy, the patient specific prosthesis components, and the patient-specific guides and templates can be manufactured using various CAD programs and/or software available, for example, by Materialise USA, Plymouth, Mich.

Thus, the patient-specific alignment guide 16 can be generally formed using computer modeling based on the patient's 3-D anatomic image and can have a patient-specific, inner engagement surface 18 that is made to conformingly contact and match a three-dimensional image of the patient's bone surface 14 (with or without cartilage or other soft tissue), by the computer methods discussed above.

It will be appreciated that the guide surfaces 20a, 20b can be of any suitable type, and the guide surfaces 20a, 20b can be custom made and patient-specific as well. For example, the guide surfaces 20a, 20b can be guiding bores or cannulated guiding posts or cannulated guiding extensions or receptacles that can be used for supporting or guiding other instruments, such as drill guides, reamers, cutters, cutting guides and cutting blocks or for inserting pins or other fasteners according to a surgeon-approved pre-operative plan.

The patient-specific alignment guide 16 and the associated cutting guide 26 can be structured to provide or define a clearance for tendons, ligaments or other tissues associated with the joint. In the exemplary illustrations of FIGS. 1-5, various alignment guides 16 and cutting guides 26 can be structured to have specific geometric features for avoiding a tendon associated with the femur or tibia of the knee joint, while enabling the placement of a cutting device (e.g., blade, drill bit, etc.) as close to the tendon as determined by the surgeon and while maintaining an alignment relative to the joint as determined by the pre-operative surgical plan.

Furthermore, the patient-specific alignment guide 16 can include one or more openings and/or guiding receptacles, which define the guide surfaces 20a, 20b. The precise location of those guide surfaces 20a, 20b can be determined on the basis of a pre-operative surgical plan for locating the referencing objects 22 and assisting in locating drilling and/or cutting instruments for resecting and shaping the joint for receiving a prosthetic implant, as described in commonly-owned, co-pending in U.S. patent application Ser. No. 11/756,057, filed on May 31, 2007, incorporated herein by reference.

Referring further to FIGS. 1 and 2, the referencing objects 22 will be discussed in greater detail. As shown, the referencing objects 22 can each be a pin 23a, 23b with a substantially straight axis. The pins 23a, 23b can be made out of rigid, metallic material. It will be appreciated, however, that the referencing objects 22 can be of any other suitable type without departing from the scope of the present disclosure.

To fix the pins 23a, 23b to the femur 13, each pin 23a, 23b can be guided by the respective guide surface 20a, 20b of the alignment guide 16 and driven partially into and fixed to the femur 13. Specifically, the pins 23a can be guided by the respective guide surfaces 20a into the anterior surface of the femur 13, and the pins 23b can be guided by the respective guide surfaces 20b into the distal end of the femur 13. The pins 23a, 23b can be driven into the femur 13 using a hammer or other similar tool (FIG. 2). Once the pins 23a, 23b are each fixed to the femur 13, the alignment guide 16 can be slid off of the pins 23a, 23b and removed to substantially expose the femur 13. For instance, the pins 23b can be removed from the femur 13, leaving holes from the pins 23b within the femur 13, and the alignment guide 16 can be slid off the pins 23a, 23b, leaving the pins 23a attached to the femur 13.

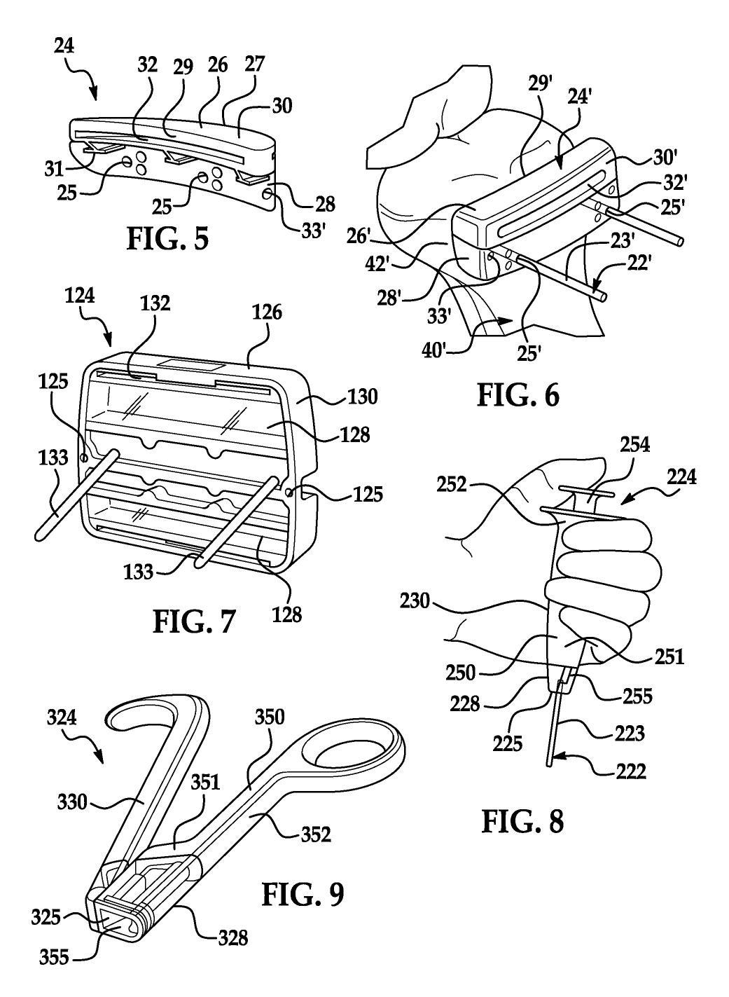

Next, referring to FIGS. 1 and 3-5, the instrument 24 will be discussed in greater detail. As mentioned above, the instrument 24 can be a resection block or cutting guide 26 for cutting a portion of the femur 13. As shown in FIGS. 4 and 5, the cutting guide 26 can be generally block-shaped and can include a first surface 27 and a second surface 29. The second surface 29 can have a concave curvature. Also, the cutting guide 26 can include a first portion 28 and a second portion 30 that are abutted and fixedly attached together. For instance, the second portion 30 can include one or more T-shaped projections 31, and the first portion 28 can encompass or otherwise receive the projections 31 to fixedly attach the first and second portions 28, 30. In some embodiments, the first and second portions 28, 30 can be formed and fixed together via a molding process (e.g., overmolding or insert molding) such that the projections 31 are embedded within the material of the first portion 28. In other embodiments, the first and second portions 28, 30 can be fixed via adhesives, fasteners, or via other means.

The reference openings 25 of the cutting guide 26 can be disposed adjacent the first portion 28. For instance, the reference openings 25 can be through holes (as shown) defined in the first portion 28. In other embodiments, the reference openings 25 can be defined in the second portion 30, directly adjacent the first portion 28. Each of the reference openings 25 can have a respective straight axis, and the reference openings 25 can extend continuously between the first and second surfaces 27, 29. In the embodiment shown, the first portion 28 can include two groups of three reference openings 25; however, it will be appreciated that the first portion 28 can include any number of reference openings 25, and the reference openings 25 can be disposed on any portion of the cutting guide 26. Also, the reference openings 25 can be of another suitable type, such as grooves, notches, etc. As mentioned above, the reference openings 25 can be sized so as to receive respective ones of the pins 23a, 23b.

The first portion 28 can also include fastening holes 33. As shown, the first portion 28 can include two fastening holes 33, each disposed on opposite edges of the first portion 28. The fastening holes 33 can axially extend at an acute angle relative to the reference openings 25. As will be discussed, the fastening holes 33 can receive fasteners (screws, etc.) separate from the pins 23a, 23b to further secure the cutting guide 26 to the femur 13. It will be appreciated that the first portion 28 can include any suitable number of fastening holes 33.

The second portion 30 of the cutting guide 26 can include a cutting guide surface 32. As shown, the cutting guide surface 32 can be the inner surface of a narrow, elongated slot; however, the cutting guide surface 32 can be an outer, peripheral surface of the cutting guide 26, an inner surface of a through-hole, or any other suitable surface. The cutting guide surface 32 can also be substantially flat or curved in two or three dimensions. As shown in FIG. 3, the cutting guide surface 32 can be used for guiding movement of a cutting device 34, such as a saw, a drill bit, etc., for cutting (e.g., resecting) the femur 13.

In some embodiments, the cutting guide 26 is non-patient-specific. In other words, the cutting guide 26 is generically shaped and sized for use among a plurality of different patients and different patient anatomies. Specifically, the peripheral surfaces (e.g., first surface 27 and second surface 29) as well as the interior surfaces (e.g., cutting guide surface 32, inner surfaces of the reference openings 25) can be generic to different anatomies of different patients. Thus, the cutting guide 26 can be used to resect the respective femur 13 of a patient, then sterilized (e.g., by autoclaving), and then re-used to resect the femur 13 of a different patient. Accordingly, while the patient-specific alignment guide 16 might be more labor-intensive to fabricate for the particular patient, the cutting guide 26 can be non-patient-specific and relatively inexpensive to fabricate. Also, because the cutting guide 26 is non-patient-specific, the cutting guide 26 can be a multiple-use component; however, the cutting guide 26 could also be a single-use, disposable component.

The second portion 30 can be made out of a high-strength, rigid metallic or polymeric material. The second portion 30 can be opaque. On the other hand, the first portion 28 can be transparent (light transmissive, translucent, etc.). For instance, the first portion 28 can be made out of a substantially clear, transparent polymeric material. In other embodiments, the first portion 28 can include a colored or tinted, but highly light transmissive material. Moreover, in some embodiments, the entire cutting guide 26 can be transparent. In some embodiments, the first portion 28 can be made of polycarbonate or acrylic material, and the second portion 30 can be made of stainless steel or other opaque metals or polymers. Thus, the cutting guide surface 32 can guide and support the cutting device 34 because the cutting guide surface 32 is formed on the higher-strength (e.g., metallic) second portion 30. For instance, if the second portion 30 is metallic, then the second portion 30 can withstand friction and other loads on the guide surface 32 without melting, chipping, or otherwise deteriorating. Meanwhile, the pins 23a, 23b can be viewed through the transparent first portion 28 when attempting to align the pins 23a, 23b with the reference openings 25.

The reference openings 25 can be entirely formed and defined within the transparent first portion 28 such that the user can look substantially along or slightly transverse to the axis of the openings 25, through the first portion 28, to see the pins 23a, 23b on the opposite side of the cutting guide 26. As such, the user can more easily judge how far the pins 23a, 23b are out-of-alignment with the respective reference openings 25 while attempting to move the pins 23a, 23b into alignment with the reference openings 25.

The system 10 can be used in a minimally invasive manner or, in particular, in a series of minimally invasive surgical steps. For instance, to use the system 10, an incision (not shown) can be made in the patient's skin (not shown), and the femur 13 can be prepared in a known manner such that the alignment guide 16 can be nested thereto. Then, one or more of the pins 23a, 23b can be driven into the femur 13 while being guided by the guide surfaces 20a, 20b of the alignment guide 16. As such, the pins 23a, 23b can be disposed in the predetermined orientation relative to the femur 13 (e.g., the axes of the pins 23a, 23b can establish a reference coordinate system for placement of the cutting guide 26). Moreover, in some embodiments, one or more of the guide surfaces 20a, 20b can guide a drill bit for drilling a hole in the femur 13, and a pin 23a, 23b can later be inserted into the hole to establish the reference coordinate system.

Next, the cutting guide 26 can be positioned onto the pins 23a, 23b by sliding the cutting guide 26 onto the pins 23a, 23b. In the embodiment shown in FIG. 3, there are only two pins 23a extending from the anterior of the femur 13, and the cutting guide 26 slides over these pins 23a such that the pins 23a are received in a corresponding pair of the reference openings 25. If necessary, additional fasteners (not shown) can be positioned through the fastening holes 33 and into the femur 13 to further secure the cutting guide 26. Also, if necessary, the cutting guide 26 can be adjusted relative to the femur 13 by receiving the pins 23a in a different pair of reference openings 25.

As discussed above, the cutting guide 26 can be conveniently aligned with and placed on the pins 23a because the surgeon can view the pins 23a through the first portion 28 of the cutting guide 26, from the first surface 27 through to the second surface 29. The surgeon can view the pins 23a through the first portion 28 from a viewpoint that is substantially aligned with the axes of the pins 23a and reference openings 25. The surgeon can recognize if the pins 23a are misaligned with the openings 25, because the pins 23a will be appear as such through the first portion 28 of the cutting guide 26, and the surgeon can judge how far the pins 23a are misaligned from the openings 25. Accordingly, the surgeon can make the necessary adjustments and more quickly attach the cutting guide 26 to the femur 13.

Next, as shown in FIG. 3, the cutting device 34 (e.g., resection blade, etc.) can be used to resect the distal end of the femur 13 while being guided by the cutting guide surface 32 of the cutting guide 26. In some embodiments, if the surgeon decides the cutting guide surface 32 needs to be repositioned, the surgeon can remove the cutting guide 26 from the pins 23a, 23b, move the cutting guide 26, and slide the cutting guide 26 onto the pins 23a, 23b such that the pins 23a, 23b are positioned within a different set of reference openings 25 of the cutting guide 26. For instance, the surgeon can move the cutting guide 26 and the cutting guide surface 32 proximally, distally, medially, laterally, or in any direction in this manner, depending on the relative location of the reference openings 25 and the pins 23a, 23b. Also, the surgeon can choose to resect the femur 13 in multiple locations, pausing to reposition the cutting guide 26 for each location.

Once the femur 13 has been resected, the surgical procedure can be completed. For instance, a prosthetic femoral implant can be implanted on the resected femur 13 for use within a prosthetic knee joint assembly (not shown).

As shown in FIG. 6, a second instrument 24′ is shown. The instrument 24′ can be a cutting guide 26′ used similarly to the cutting guide 26 discussed above. However, the cutting guide 26′ can be used for resecting a tibia 40′ (e.g., a proximal end thereof). Specifically, the pins 23′ can be fixed to the tibia 40′ in the same manner discussed above (i.e., using a patient-specific guide 16 that has a three-dimensionally curved surface that corresponds and nests against a corresponding tibial surface 42′). Then, the cutting guide 26′ can be positioned over the pins 23′ to fit to the tibia 40′, and the cutting device (not shown) can be used to resect the tibia 40′ while being guided by the cutting guide surface 32′ of the cutting guide 26′. Subsequently, a tibial portion of a knee prosthetic device (not shown) can be implanted on the tibia 40′.

It will be appreciated that the cutting guide 26′ can be substantially similar to the cutting guide 26 described in detail above in connection with FIGS. 1-5. In other embodiments, the cutting guide 26′ can have one or more features that are specific for resecting the tibia 40′. In both cases, the cutting guide 26′ can be at least partially transparent as described above. As such, aligning the cutting guide 26′ onto the pins 23′ can be greatly facilitated.

Referring now to FIG. 7, additional embodiments of a third instrument 124 with at least a portion 128 that is transparent is illustrated. Features that are similar to the embodiments discussed above are indicated with reference numerals increased by 100.

As shown, the instrument 124 can be a cutting guide 126 that incorporates various features of a Femoral 4-in-1 Resection Block, commercially available from Biomet, Inc. of Warsaw, Ind. Thus, the cutting guide 126 can be block-shaped. The cutting guide 126 can also include one or more projections 133 (e.g., nails, pins, etc.) that extend therefrom. The cutting guide 126 can also include one or more openings 125. The cutting guide 126 can also include one or more cutting guide surfaces 132 for cutting (resecting) the bone.

Moreover, the cutting guide 126 can include one or more transparent first portions 128 and a second portion 130. In the embodiment shown, the cutting guide 126 can include two block-shaped, transparent first portions 128, and the opaque, second portion 130 can extend about and between both the first portions 128. The cutting guide surfaces 132, the openings 125, and the projections 133 can be defined in the second portion 130.

To attach the cutting guide 126 to the femur 13, a drill bit or other tool can be guided by guide surfaces 20 of the alignment guide 16 to form openings (e.g., holes) within the femur 13 as discussed above in relation to FIGS. 1 and 2. Then, the surgeon can remove the alignment guide 16 from the femur 13, leaving the holes therein, and the projections 133 of the cutting guide 126 can be inserted into these openings. It will be appreciated that the surgeon can view the position of the projections 133 relative to the holes in the femur 13 through the transparent first portions 128 of the cutting guide 126. Accordingly, as discussed above, the surgeon can more easily align the projections 133 with the corresponding holes in the femur 13 for added convenience. Once attached, the cutting guide 126 can be further secured to the femur 13 by attaching additional fasteners through the openings 125.

Referring now to FIG. 8, additional embodiments of a fourth instrument 224 are illustrated. Features that are similar to the embodiments discussed above in relation to FIGS. 1-5 are indicated with reference numerals increased by 200.

As shown, the instrument 224 can be a grasping instrument 250 that selectively grasps the pins 223 discussed above. Specifically, the instrument 250 can include a body 251 with a handle 252, an opening 225 at one end, and a button 254 at the opposite end. The button 254 can be depressed to actuate a grasping mechanism 255, such as a chuck, which is disposed within the opening 225. Thus, the opening 225 can receive the pin 223, and the user can depress the button 254 such that the grasping mechanism 255 grasps the pin 223. In some embodiments, the grasping mechanism 255 can grasp the pin 223 via friction alone. In other embodiments, the pin 223 can have an enlarged head that the grasping mechanism 255 engages. As such, the instrument 224 can be used for pulling the pin 223 out of the femur 13, tibia 40′, or other anatomical feature.

In some embodiments, the instrument 224 can be tapered inwardly adjacent the opening 225 to help guide the pin 223 into the opening 225. Also, it will be appreciated that the opening 225 and grasping mechanism 255 can be adapted so as to receive and selectively grasp any of the various referencing objects 222, including, but not limited to the pin 223.

As shown, the body 251 can include a first portion 228 that is transparent. The body 251 can also include a second portion 230 that is opaque. The handle 252 and button 254 can be defined in the second portion 230, and the opening 225 can be defined in the first portion 228. Thus, the user can view the pin 223 through the first portion 228 to aid in aligning the pin 223 and the opening 225. Also, the user can view the pin 223 entering and extending into the opening 225 so as to ensure engagement of the pin 223 and the grasping mechanism 255. This can be much more convenient than conventional, fully opaque instruments that block the pin 223 well before the pin 223 is engaged by the instrument. Accordingly, grasping and manipulating the pins 223 can be facilitated.

Next, referring to FIG. 9, a fifth instrument 324 is illustrated. The fifth instrument 324 (another grasping instrument 350) is illustrated according to additional teachings of the present disclosure. Components that are similar to those of FIG. 8 are indicated with corresponding reference numerals increased by 100.

As shown, the grasping instrument 350 can have a scissor-like handle 352. Also, the opening 325 can be defined on a transparent first portion 328, similar to the embodiments of FIG. 8. As such, the user can view the pin (not shown) through the first portion 328 to align the pin and the opening 325. Also, the user can view the pin entering and extending into the opening 325 so as to ensure engagement of the pin and the grasping mechanism 355. Again, this can be much more convenient than conventional grasping instruments, which block the view of the pin well before the pin is engaged by the grasping instrument. Thus, grasping and pulling the pin can be facilitated.

The foregoing discussion discloses and describes merely exemplary arrangements of the present teachings. Furthermore, the mixing and matching of features, elements and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the teachings of the invention without departing from the essential scope thereof. One skilled in the art will readily recognize from such discussion, and from the accompanying drawings and claims, that various changes, modifications and variations can be made therein without departing from the spirit and scope of the present teachings as defined in the following claims.

What is claimed is:

1. A cutting guide for cutting an anatomical feature comprising:
a first portion with a cutting guide surface that guides a cutting device when cutting the anatomical feature, the first portion being opaque; and
a second portion that is coupled to the first portion, the second portion being transparent to view the anatomical feature through the second portion and to orient the cutting guide relative to the anatomical feature.

2. The cutting guide of claim 1, wherein at least one of the first portion and the second portion defines an opening that receives a referencing object that is coupled to the anatomical feature, the referencing object viewable through the second portion to align the referencing object with the opening and receive the referencing object within the opening to orient the cutting guide in a reference orientation relative to the anatomical feature.

3. The cutting guide of claim 2, wherein the second portion defines the opening.

4. The cutting guide of claim 2, further comprising a projection extending from the cutting guide that is viewable through the second portion, the projection operable to be received within an opening in the anatomical feature to orient the cutting guide in a reference orientation relative to the anatomical feature.

5. The cutting guide of claim 1, wherein the first portion is made out of a metallic material.

6. The cutting guide of claim 1, wherein the cutting guide surface is a slot that is operable to guide a cutting device when cutting the anatomical feature.

7. The cutting guide of claim 1, wherein at least one of the first portion and the second portion includes a projection that is embedded within the other of the first portion and the second portion to thereby fixedly couple the first and second portions together.

8. The cutting guide of claim 1, wherein the first and second portions each include a plurality of outer peripheral surfaces, and wherein the plurality of outer peripheral surfaces are each entirely non-patient-specific and useable for cutting the same anatomical feature of different patients.

9. A system for orienting objects relative to an anatomical feature of a patient, the system comprising:
a guide including a reference guide surface;
a referencing object that is guided by the reference guide surface toward the anatomical feature to be fixed to the anatomical feature in a reference orientation relative to the anatomical feature; and
an instrument with an opening and at least a portion that is transparent adjacent the opening, the referencing object viewable through the transparent portion of the instrument to align the referencing object with the opening, the opening receiving the referencing object to orient the instrument in the reference orientation relative to the anatomical feature.

10. The system of claim 9, wherein the guide has a patient-specific surface with a three-dimensional contour that nests and closely conforms to a corresponding surface of the anatomical feature to align the guide relative to the anatomical feature.

11. The system of claim 9, wherein the transparent portion defines the opening.

12. The system of claim 9, wherein the instrument is a cutting guide with a cutting guide surface that guides a cutting device when cutting the anatomical feature.

13. The system of claim 12, wherein the cutting guide includes a first portion and a second portion that are coupled together, the first portion defining the opening, the first portion being transparent, the second portion including the cutting guide surface.

14. The system of claim 13, wherein the first portion is made of a polymeric material and the second portion is made of a metallic material.

15. The system of claim 12, wherein the cutting guide is a resection guide for resecting a portion of at least one of a femur and a tibia.

16. The system of claim 9, wherein the instrument is a grasping instrument that selectively grasps the referencing object for removing the referencing object from the anatomical feature.

17. The system of claim 9, wherein the opening is a through hole through which the referencing object extends through the instrument.

18. The system of claim 9, wherein the instrument includes a plurality of outer peripheral surfaces, and wherein the plurality of outer peripheral surfaces are each entirely non-patient-specific and useable for the same anatomical feature of different patients.

19. A method of orienting objects relative to an anatomical feature of a patient, the method comprising:
coupling a guide to the anatomical feature;
guiding a referencing object toward the anatomical feature using a reference guide surface of the guide to establish the referencing object in a reference orientation relative to the anatomical feature;
providing an instrument with an opening and at least a portion that is transparent adjacent the opening;
orienting the instrument in the reference orientation relative to the anatomical feature by viewing the referencing object through the portion of the instrument and receiving the referencing object within the opening of the instrument.

20. The method of claim 19, wherein coupling the guide includes nesting a three-dimensionally contoured, patient-specific surface of the guide to a corresponding surface of the anatomical feature to align the patient-specific guide relative to the anatomical feature.

21. The method of claim 19, wherein orienting the instrument includes aligning the referencing object within the opening while viewing the referencing object through the portion of the instrument.

22. The method of claim 19, further comprising guiding a cutting device with a cutting guide surface of the instrument and cutting the anatomical feature.

23. The method of claim 22, further comprising resecting a portion of at least one of a femur and a tibia with the cutting device.

24. The method of claim 19, further comprising grasping the referencing object with the instrument.

25. The method of claim 24, further comprising removing the referencing object using the instrument while the instrument grasps the referencing object.

26. A system for orienting objects relative to an anatomical feature of a patient, the system comprising:
a patient-specific guide having a patient-specific surface with a three-dimensional contour that nests and closely conforms to a corresponding surface of the anatomical feature to align the patient-specific guide relative to the anatomical feature, the patient-specific guide also including a reference guide surface;
a pin that is guided by the reference guide surface toward the anatomical feature to be fixed to the anatomical feature in a reference orientation relative to the anatomical feature; and
a cutting guide with an opening, the cutting guide including a metallic portion that defines a cutting guide surface that guides a cutting device for cutting the anatomical feature, the cutting guide also including a transparent portion that defines the opening, the pin being viewable through the transparent portion to align the pin with the opening, the opening receiving the pin to orient the cutting guide in the reference orientation relative to the anatomical feature.
